(19) United States
(12) Patent Application Publication
(10) Pub. No.: US 2013/0023823 A1
(43) Pub. Date: Jan. 24, 2013

(54) IMPLANTABLE THERAPEUTIC DEVICE AND METHODS OF MAKING

(71) Applicants: Nicholas Edward Simpson, Alachua, FL (US); Mark John Beveridge, Gainesville, FL (US); Nelly Aline Volland, Salt Lake City, UT (US)

(72) Inventors: Nicholas Edward Simpson, Alachua, FL (US); Mark John Beveridge, Gainesville, FL (US); Nelly Aline Volland, Salt Lake City, UT (US)

(73) Assignee: UNIVERSITY OF FLORIDA RESEARCH FOUNDATION INC., GAINESVILLE, FL (US)

(21) Appl. No.: 13/583,648

(22) PCT Filed: Mar. 10, 2011
(86) PCT No.: PCT/US11/27921

§ 371 Date: Sep. 10, 2012

Related U.S. Application Data
(60) Provisional application No. 61/312,522, filed on Mar. 10, 2010.
(60) Provisional application No. 61/328,254, filed on Apr. 27, 2010.

(51) Int. Cl. A61M 31/00; B29C 67/24

(52) U.S. Cl. 604/93.01; 264/241

(57) ABSTRACT

The subject invention pertains to an implantable therapeutic device for treating diabetes and methods of making. Upon implantation, the present device secretes insulin in response to blood glucose levels, exquisitely regulates blood glucose levels, reduces hyperglycemia, and includes β-cell regeneration in the host. It is useful for treating or ameliorating diabetes or diabetic conditions of a subject, including but not limited to, type 1 diabetes mellitus, hyperglycemia, impaired glucose tolerance, insulin deficiency, elevated glucose levels, and insulin resistance.

### CROSS-REFERENCE TO RELATED APPLICATIONS

This application claims the benefit of U.S. Provisional Application Ser. No. 61/312,522, filed Mar. 10, 2010, and U.S. Provisional Application Ser. No. 61/328,254, filed Apr. 27, 2010, the disclosures of which are hereby incorporated by reference in their entireties, including all figures, tables and amino acid or nucleic acid sequences.

### GOVERNMENT SUPPORT

This invention was made with Government support under the National Institutes of Health (NIH) and the National Institute of Diabetes and Digestive and Kidney Diseases (NIDDK) Grant No. NIDDK RO1 47858. The Government has certain rights in the invention.

### BACKGROUND OF THE INVENTION

Insulin, a small protein hormone produced by β-cells in the pancreatic islets of Langerhans, plays an essential role for maintaining normal glucose levels (normoglycemia) and regulating metabolic processes in the body. Insulin stimulates the cellular uptake and metabolism of glucose and amino acids, induces glycogenesis and lipogenesis, and suppresses gluconeogenesis.

Diabetes mellitus develops when insulin is not sufficiently produced or recognized by the body. Dysregulation of blood-glucose levels triggers severe short-term and long-term tissue damages in the eye, kidney, heart, and vasculature. It is estimated that diabetes will affect over 330 million people by 2025 (King et al., 1998). Roughly 8% of diabetics have the insulin-dependent variety of diabetes (also known as type I diabetes), which occurs due to the destruction of the insulin-secreting β-cells.

A common treatment method for type 1 diabetes involves regular injections of insulin to a patient in order to control blood-glucose levels. However, injections of insulin cannot mimic the exquisite insulin secretory process in response to changing glucose levels offered by healthy β-cells. Consequently, insulin injections may not prevent the development of severe complications leading to eye, kidney, heart, and vascular diseases and even a shortened lifespan. Another approach for treating type 1 diabetes involves replacing β-cells through pancreatic or islet transplantation (Robertson et al., 1992; Sutherland 1996; Shapiro et al., 2000). However, this approach is significantly limited by the scarcity of donor tissues, possible immune rejections, and the need of continuous administration of immunosuppressive medications.

Therefore, a substantial need exists for improved treatment methods that would allow exquisite regulation of blood glucose levels without the need of immunosuppressive medications.

### BRIEF SUMMARY OF THE INVENTION

The aforementioned need is satisfied by the present implantable therapeutic device that secretes insulin in response to blood glucose levels, exquisitely regulates blood glucose levels, reduces hyperglycemia, and/or includes β-cell regeneration in the host. It is useful for treating or ameliorating diabetes or diabetic conditions of a subject, including but not limited to, type-I diabetes mellitus, hyperglycemia, impaired glucose tolerance, insulin deficiency, elevated glucose levels, and insulin resistance. The device comprises:

a) an outer housing made of an insulating material, such as polydimethylsiloxane (PDMS), wherein at least part of the outer housing is an open hollow cavity;

b) an inner housing situated inside the open hollow cavity of said outer housing, wherein the inner housing is a substantially enclosed structure;

c) cells of interest; and

d) alginate beads encapsulating cells of interest, and wherein the cell-encapsulated alginate beads are enclosed inside the inner housing.

Further provided are methods of making the present implantable therapeutic device, comprising:

a) providing liquid polydimethylsiloxane (PDMS);

b) forming the liquid polydimethylsiloxane (PDMS) into an outer housing wherein at least part of the outer housing is an open hollow cavity;

c) providing a liquid alginate having guluronic acid concentration of 15%-45% by weight percentage;

d) dispersing cells of interest within the liquid alginate mixture to form an alginate-cell suspension;

e) forming alginate beads encapsulating cells of interest from the alginate-cell suspension by gelling with a divalent cation (such as, but not limited to Mg++, Ca++, Ba++);

f) mixing the alginate beads with an alginate mixture guluronic acid content of about 40%-80% by weight percentage; and

g) filling the outer housing with the alginate beads/alginate mixture and spinning the outer housing while the alginate solidifies to form an inner housing by gelling with a divalent cation (such as, but not limited to Mg++, Ca++, Ba++); wherein the inner housing is situated inside the open hollow cavity of the outer housing, and wherein the alginate beads encapsulating cells of interest are enclosed inside the inner housing.

In an embodiment, the present therapeutic device, more specifically the outer housing, may further comprise an additional device, such as NMR coils, thereby allowing for non-invasive observation and monitoring of the host responses and implanted tissue viability and function upon implantation.

### BRIEF DESCRIPTION OF THE DRAWINGS

FIGS. 1A-1B depict various embodiments of the implantable therapeutic device of the present invention. FIG. 1A is an outer housing without an NMR coil. FIG. 1B is an outer housing that includes an NMR coil.

FIG. 2 depicts an embodiment of the implantable therapeutic device of the present invention, comprising an outer housing, an inner housing, and cell-encapsulating alginate beads enclosed inside said inner housing. In this Figure, there are three rings of differing inner diameter to create the desired resultant PDMS inner core shape, and the top ring is removed. Alginate beads (approx. 400 micron diameter) which can contain insulin-secreting cells are stained. These beads are entrapped in alginate that is contained within the outer (clear) bioinert material.

FIG. 3(A) shows the outer housing (no alginate inserted) of the present device, wherein the outer housing further contains an NMR coil tuned to This coil can be inductively-coupled to a matched and tuned surface coil such that NMR data from only the tissues contained within the device are obtained. FIG. 3(B) shows an NMR image from an implanted inductively-coupled coil of an acellular alginate beads-containing device (spin-echo sequence; TR=2.5 s; TE=10 ms; 1 mm slice thickness, 1 average, FOV=2 cm×2 cm; 256×256 matrix; respiratory gating applied). The white circle indicates the outer edge. Individual beads can be discerned in this in vivo image.

FIG. 4 shows the average fasting blood glucose levels of diabetic recipients (here, mice) of the implantable therapeutic device of the present invention. Blood glucose levels of diabetic mice implanted with our bioartificial pancreas on day 4. Triangles: acellular (n=4); Squares: device with βTC-tet cells (n=16); Diamonds: data from a ‘cured’ animal. The normoglycemic level (average+/−st. dev.) for this mouse strain is shown in the grey band.

At day 38, the implant on the ‘cured’ animal was removed.

FIG. 5 depicts the histology of pancreas taken from animal whose construct was removed. Slide shows islet (cytoplasm stained red, insulin) and mitotic cells (nuclei stained dark brown, Ki67). The slide demonstrates this animal had regeneration of the islets, sufficient to sustain life. However, without the implant, the animal would have succumbed to hyperglycemia before sufficient regeneration would have occurred.

FIG. 6 shows a Kaplan-Meier survival curve of diabetic mice. The graph shows mice rendered diabetic by 62.5 mg/kg alloxan (n=4, no animal surviving at day 15); 75 mg/kg alloxan (n=15, no animals surviving at day 5) and given no treatment (or acellular constructs). These mice died rapidly, with half the recipients of the higher diabetic inductor dose surviving only ˜3 days. Diabetic mice given cellular constructs (βTC-tet cells entrapped in alginate beads in an alginate core, as described in the procedures) survived much longer: induction with alloxan, 62.5 mg/kg (n=17, 70% of animals surviving at day 30); or 75 mg/kg (n=13, 60% of animals surviving at day 30).

FIGS. 7A, 7B, 8A and 8B illustrate an exemplary shape suitable for an outer housing (10) as described herein. This ring shape contains flanges (34) at both the upper and lower edge of the ring structure (see cross section in FIG. 10A) that can support an inner housing or a support member (15). A support member (15) can be inserted into the outer housing to support the formation of an inner housing if desired. The inner (38) and outer (30) surfaces of the outer housing are also depicted.

FIGS. 9A and 9B illustrate another exemplary shape suitable for an outer housing (10). In this case, the outer housing contains a single flange (34) that can support an inner housing or a support member (15) upon which an inner housing can be formed. The inner (38) and outer (30) surfaces of the outer housing are also depicted.

FIGS. 10A and 10B illustrate cross-sectional views of the exemplary outer housings depicted in FIGS. 7A, 7B, 8A, 8B, 9A and 9B. The thickness of a flange (34) can be of the same or of a different thickness of a wall of the outer housing (defined between the inner (38) and outer (30) wall). FIG. 10A depicts an outer housing with flanges at the upper and lower edge of the ring structure. FIG. 10B depicts an outer housing containing a single flange. The angle at which the flanges are attached or formed, with relation to the upper and/or lower edge of a shape, may be perpendicular or substantially perpendicular to the walls formed by the inner and outer surfaces of the shape (e.g., a ring structure). A substantially perpendicular flange, with relation to the upper and/or lower edge of a shape, need only be capable of supporting an inner housing or a support member. Additionally, imaging devices (e.g., tuned NMR coils) can be embedded in (or attached to) the wall of an outer housing.

FIG. 11. ¹⁹F image of 5% PFCE in alginate beads in vitro using a spin-echo pulse sequence (TR=1000 ms, TE=12 ms, 20 averages, 64-by-64 matrix) with a 4×4 cm² view. ¹⁹F imaging and spectroscopy with NMR allows for non-invasive oxygen level determination. This image demonstrates the utility of including a coil in the construct to obtain critical measures of key nuclei to yield metabolic information.

### DETAILED DISCLOSURE OF THE INVENTION

The present invention provides an implantable therapeutic device for maintaining normoglycemia in a diabetic subject and methods of making. The therapeutic device houses insulin-secreting cells entrapped in alginate beads, and when implanted, the cells within the device can secrete insulin in response to the recipient's glucose levels Therefore, the present implantable therapeutic device is useful for treating or ameliorating diabetes or diabetic conditions of a subject, including but not limited to, type-I diabetes mellitus, hyperglycemia, impaired glucose tolerance, insulin deficiency, elevated glucose levels, and insulin resistance. The implantable device provided herein can also be used to provide for the delivery of other therapeutic agents, such as interferons, interleukins, tumor necrosis factors, growth hormones and other cytokines or endocrine molecules to a subject (e.g., an animal or a human).

Non-limiting examples of endocrine molecules that can be delivered using the devices disclosed herein include: norepinephrine; epinephrine; dopamine; thyroid hormones, such as 3,5,3′-triiodothyronine (T3) and 3,5,3′,5′-tetraiodothyronine (thyroxine, T4); follicle stimulating hormone (FSH), thyroid stimulating hormone (TSH), endorphins, luteinizing hormone (LH), prolactin, growth hormone, parathyroid hormone, leptin, ghrelin, cortisol, corticosterone, testosterone, melanocyte stimulating hormone, adrenocorticotropic hormone (ACTH), oxytocin, antidiuretic hormone, aldosterone, dehydroepiandrosterone (DHEA), DHEA sulfate (DHEA-S) glucocorticoids, mineralocorticoids, androgens, estrogens or progestagens. Such endocrine molecules can be secreted from isolated tissue cells that secrete these molecules (e.g., cells from the thyroid, parathyroid, adrenal or pituitary tissues) or by cells genetically engineered to secrete such endocrine molecules and are used to treat thyroid, parathyroid, adrenal or pituitary disorders associated with the underproduction of such endocrine molecules.

The structure of the implantable therapeutic device

One aspect of this invention provides a surgically implantable therapeutic device for treating diabetes, comprising:

a) an outer housing made of a material (for example, polydimethylsiloxane (PDMS)), wherein at least part of the outer housing is an open hollow cavity;

b) an inner housing situated inside the open hollow cavity of said outer housing, wherein the inner housing is a substantially enclosed structure (e.g., a solid disc);

c) cells of interest; and

d) alginate beads encapsulating cells of interest, and wherein the cell-encapsulated alginate beads are enclosed inside the inner housing.

In a specifically exemplified embodiment, the outer housing is in a ring shape made of material comprising polydimethylsiloxane (PDMS). There are three rings: the top ring and the bottom ring are about 1 mm thick and 14 mm in diameter, and the center ring is about 2 mm thick, 14 mm in outer diameter, and 12 mm in inner diameter. The PDMS ring structure creates a core into which the alginate inner housing can be situated. Because alginate does not stick to PDMS, the center slab inner diameter (—11-12 mm) is slightly larger than the outer slabs inner diameter (−9.5 mm). This creates a center ledge, which traps the gelled alginate into the outer housing. The inner housing is a hard alginate 10 mm-diameter core, situated within the 14 mm-diameter polydimethylsiloxane (PDMS) ring. Soft alginate beads of about 400 micron in diameter, entrapped with insulin-secreting cells, are situated inside the inner hard alginate core. Both polydimethylsiloxane (PDMS) and alginate are biologically inert, and thus do not elicit substantially adverse immune responses from the host.

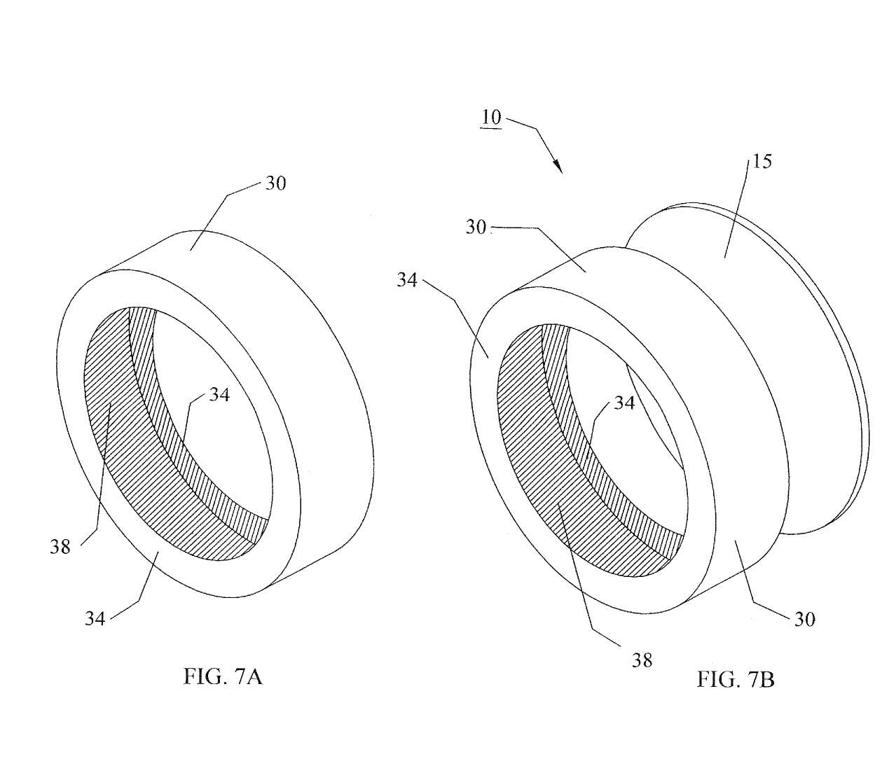

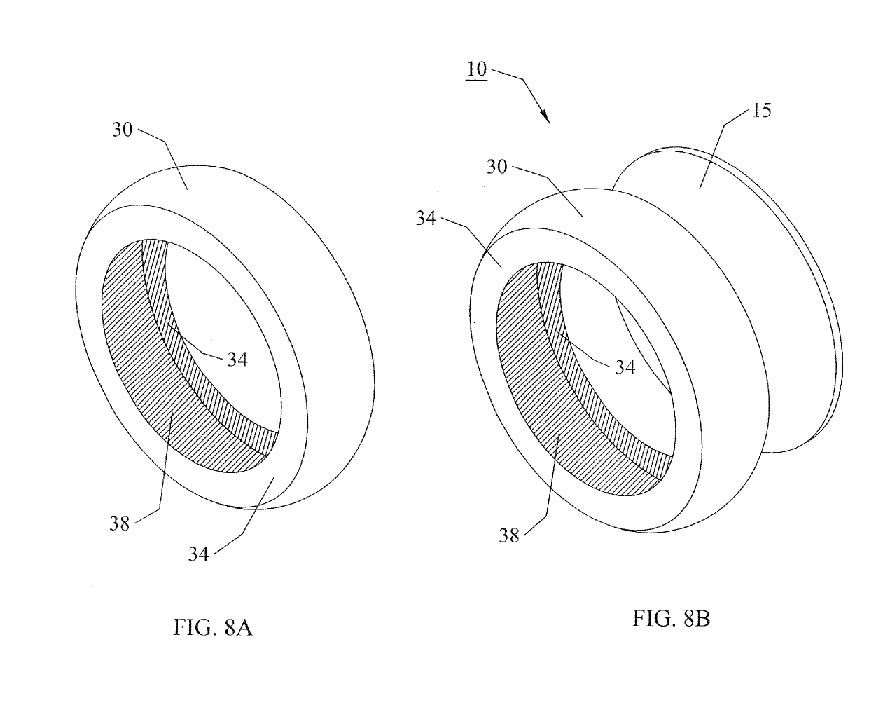

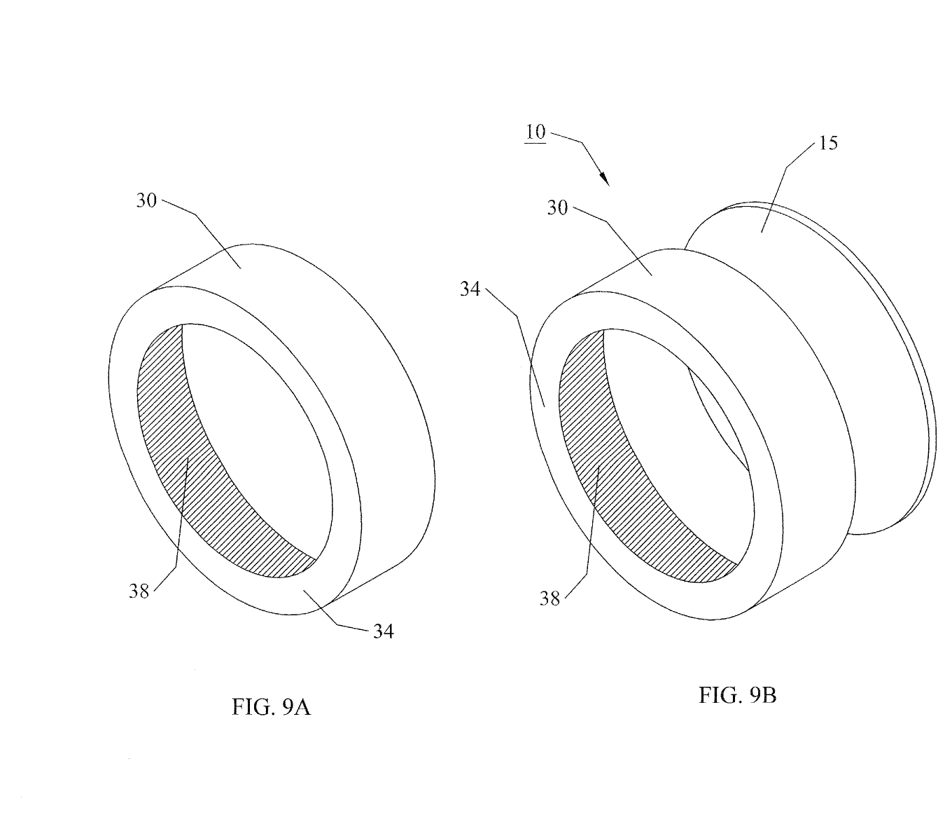

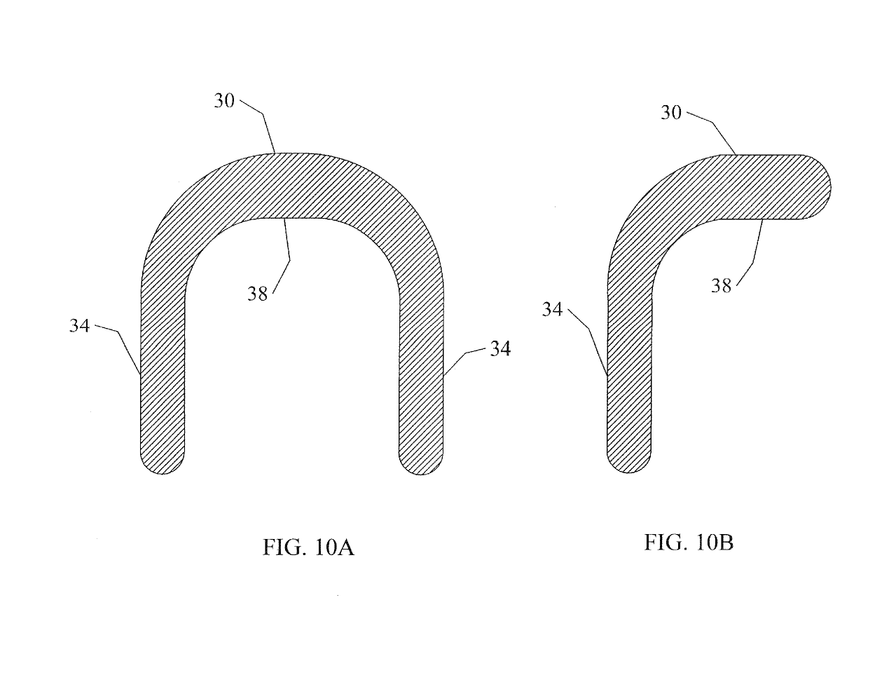

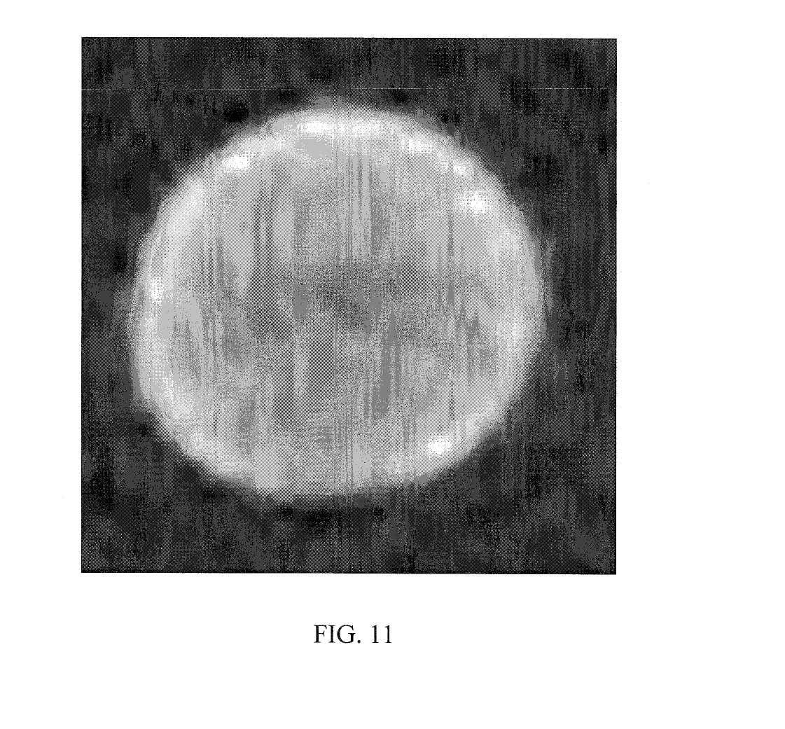

In another embodiment, the outer housing can be constructed (or molded) in a shape (e.g., a ring) that contains flanges onto which the inner housing may be situated (see, for example, FIGS. 7-8). As illustrated in those figures, the flange(s) may be on one or both sides of the shape that forms the outer housing. In certain aspects of this embodiment, a support member may be situated into the ring structure such that it is supported by the lower flange of the outer housing and the inner housing may be formed on the support member. The support member may be made of a permeable or impermeable material at the option of the fabricator; however, for optimal diffusion of a therapeutic agent produced by cells encapsulated within the inner housing, it is preferable that the support member be permeable (e.g., a surgical mesh that is biologically inert).

The outer housing may be of any shape or structure adapted to situate the inner housing. In certain embodiments, the outer housing may be any shape that has an open hollow cavity, for example, a ring, a coil, a hollow truncated cone, a hollow sphere, a hollow cylinder, a tube, a hollow cube, a hollow inflated oval, or any hollow irregular geometric shape. Preferably, the shape of the outer housing conforms, at least in part, to the shape of the inner housing and other therapeutic devices the outer housing may contain and the outer housing may, optionally, have one or two flanges that support the inner housing or that are able to provide support to a support member for the inner housing that is inserted into the outer housing. Further, the outer housing is preferably of a shape that allows for easy manipulation, such as during implantation and retrieval by a health care provider. Additionally, the structure of the outer housing does not damage tissues and internal organs of a recipient. Additionally, the outer housing may be made of a rigid or flexible material. The use of a flexible material allows the fabricator to insert a permeable or impermeable support member into the outer housing upon which an inner housing can be fabricated.

In certain embodiments, the outer housing is comprised of a ring or a plurality of rings having an inner diameter of about 0.1 mm, 0.2 mm, 0.3 mm, 0.4 mm, 0.5 mm, 0.6 mm, 0.7 mm, 0.8 mm, 0.9 mm, 1.0 mm, 1.5 mm, 2.0 mm, 2.5 mm, 3.0 mm, 3.5 mm, 4.0 mm, 4.5 mm, 5.0 mm. 5.5 mm, 6.0 mm, 6.5 mm, 7.0 mm, 7.5 mm, 8.0 mm, 8.5 mm, 9.0 mm, 9.5 mm, 10.0 mm, 10.5 mm, 11.0 mm, 11.5 mm, 12.0 mm, 12.5 mm, 13.0 mm, 13.5 mm, 14.0 mm, 14.5 mm, 15.0 mm, 15.5 mm, 16.0 mm, 16.5 mm, 17.0 mm, 17.5 mm, 18.0 mm, 18.5 mm, 19.0 mm, 19.5 mm, 20.0 mm, 20.5 mm, 21.0 mm, 21.5 mm, 22.0 mm, 22.5 mm, 23.0 mm, 23.5 mm, 24.0 mm, 24.5 mm, 25.0 mm, 25.5 mm, 26.0 mm, 26.5 mm, 27.0 mm, 27.5 mm, 28.0 mm, 28.5 mm, 29.0 mm, 29.5 mm, 30.0 mm, 35.0 mm, or 40.0 mm. In alternative embodiments, the inner diameter of the outer housing is about 0.1 mm-40.0 mm, 0.5 mm-35.0 mm, 1.0 mm-30.0 mm, 1.5 mm-25.0 mm, 2.0 mm-20.0 mm, 4.0 mm-15.0 mm, or 5.0 mm-10.0 mm.

In certain embodiments, the outer housing is comprised of a ring or a plurality of rings having an outer diameter of about 0.1 mm, 0.2 mm, 0.3 mm, 0.4 mm, 0.5 mm, 0.6 mm, 0.7 mm, 0.8 mm, 0.9 mm, 1.0 mm, 1.5 mm, 2.0 mm, 2.5 mm, 3.0 mm, 3.5 mm, 4.0 mm, 4.5 mm, 5.0 mm, 5.5 mm, 6.0 mm, 6.5 mm, 7.0 mm, 7.5 mm, 8.0 mm, 8.5 mm, 9.0 mm, 9.5 mm, 10.0 mm, 10.5 mm, 11.0 mm, 11.5 mm, 12.0 mm, 12.5 mm, 13.0 mm, 13.5 mm, 14.0 mm, 14.5 mm, 15.0 mm, 15.5 mm, 16.0 mm, 16.5 mm, 17.0 mm, 17.5 mm, 18.0 mm, 18.5 mm, 19.0 mm, 19.5 mm, 20.0 mm, 20.5 mm, 21.0 mm, 21.5 mm, 22.0 mm, 22.5 mm, 23.0 mm, 23.5 mm, 24.0 mm, 24.5 mm, 25.0 mm, 25.5 mm, 26.0 mm, 26.5 mm, 27.0 mm, 27.5 mm, 28.0 mm, 28.5 mm, 29.0 mm, 29.5 mm, 30.0 mm, 35.0 mm, 40.0 mm, 45.0 mm, or 50.0 mm. In alternative embodiments, the outer diameter of the outer housing is about 0.1 mm-50.0 mm, 0.5 mm-45.0 mm, 1.0 mm-40.0 mm, 1.5 mm-35.0 mm, 2.0 mm-30.0 mm, 4.0 mm-25.0 mm, 5.0 mm-20.0 mm, or 7.0 mm-15.0 mm.

In certain embodiments, the outer housing is comprised of a ring or a plurality of rings having a thickness of about 0.1 mm, 0.2 mm, 0.3 mm, 0.4 mm, 0.5 mm, 0.6 mm, 0.7 mm, 0.8 mm, 0.9 mm, 1.0 mm, 1.1 mm, 1.2 mm, 1.3 mm, 1.4 mm, 1.5 mm, 1.6 mm, 1.7 mm, 1.8 mm, 1.9 mm, 2.0 mm, 2.1 mm, 2.2 mm, 2.3 mm, 2.4 mm, 2.5 mm, 2.6 mm, 2.7 mm, 2.8 mm, 2.9 mm, 3.0 mm, 4.1 mm, 4.2 mm, 4.3 mm, 4.4 mm, 4.5 mm, 4.6 mm, 4.7 mm, 4.8 mm, 4.9 mm, or 5.0 mm. In alternative embodiments, the thickness of the ring is about 0.1 mm-5.0 mm, 0.3 mm-4.5 mm, 0.5 mm-4.0 mm, 0.7 mm-3.5 mm, 0.9 mm-3.0 mm, 1.0 mm-2.5 mm, or 1.5 mm-2.0 mm.

The inner housing may be of any shape or structure adapted to be situated inside the outer housing and contain alginate beads entrapped with cells of interest. In certain embodiments, the shape of the inner housing may be, for example, a coil, a cone, a sphere, a cylinder, a cube, a tetrahedron, an inflated oval, or any irregular geometric shape. In a specific embodiment, the shape of the inner housing is a 10 mm-diameter core.

In certain embodiments, the diameter of the inner housing is about 0.1 mm, 0.2 mm, 0.3 mm, 0.4 mm, 0.5 mm, 0.6 mm, 0.7 mm, 0.8 mm, 0.9 mm, 1.0 mm, 1.5 mm, 2.0 mm, 2.5 mm, 3.0 mm, 3.5 mm, 4.0 mm, 4.5 mm, 5.0 mm, 5.5 mm, 6.0 mm, 6.5 mm, 7.0 mm, 7.5 mm, 8.0 mm, 8.5 mm, 9.0 mm, 9.5 mm, 10.0 mm, 10.5 mm, 11.0 mm, 11.5 mm, 12.0 mm, 12.5 mm, 13.0 mm, 13.5 mm, 14.0 mm, 14.5 mm, 15.0 mm, 15.5 mm, 16.0 mm, 16.5 mm, 17.0 mm, 17.5 mm, 18.0 mm, 18.5 mm, 19.0 mm, 19.5 mm, 20.0 mm, 20.5 mm, 21.0 mm, 21.5 mm, 22.0 mm, 22.5 mm, 23.0 mm, 23.5 mm, 24.0 mm, 24.5 mm, 25.0 mm, 25.5 mm, 26.0 mm, 26.5 mm, 27.0 mm, 27.5 mm, 28.0 mm, 28.5 mm, 29.0 mm, 29.5 mm, 30.0 mm, 35.0 mm, or 40.0 mm. In alternative embodiments, the diameter of the inner housing is about 0.1 mm-40.0 mm, 0.5 mm-35.0 mm, 1.0 mm-30.0 mm, 1.5 mm-25.0 mm, 2.0 mm-20.0 mm, 4.0 mm-15.0 mm, or 5.0 mm-10.0 mm.

Preferably, the present therapeutic device is made of, primarily, substantially biologically inert or biologically compatible materials. The term “inert,” “biologically inert” or “biologically compatible,” as used herein, refers to a substance or material that, after the normal healing period when implanted into living tissues, does not elicit substantially adverse biochemical, allergic, or immune responses and/or does not induce substantial fibrotic overgrowth. Additionally, the outer housing and the inner housing preferably comprise one or more materials capable of containing cells for a prolonged period of time, and thus prevent unwanted cell escape. Both the outer and inner housings may further comprise one or more materials exhibiting excellent mechanical strength and amenability, and thus enhance the ease of manipulation during manufacturing and implantation and explantation by a health care provider. Thus, materials having excellent biocompatibility, strength, chemical and thermal stability, and longevity to function permanently or for a long time in a recipient are preferred.

In an embodiment, the outer housing consists essentially of polydimethylsiloxane (PDMS). In alternative embodiments, the outer housing comprises materials including, but not limited to, PDMS, carbon fiber, polyethylene oxide polymers, ceramics, Teflon®, acrylonitrile, butadiene, styrene, acetates, acrylics, delrin, epoxy fiberglass, glass, kymar, mica, nylon, polyetheretherketone (PEEK), polyethylene terephthalate (PET), polypropylene, polystyrene, polyurethane, polyvinyl chloride, or any combination thereof Alternatively, the outer housing may be coated with one or more layers of biologically inert materials, using techniques well known in the art, such as chemical vapor deposition, physical vapor deposition, or sputtering.

The inner housing may be made of a biologically compatible material, comprising alginate, agar, collagen, gelatin, or any combination thereof. These can be milled or formed to the desired internal shape to allow inclusion of the alginate plug as defined earlier. In an embodiment, the inner housing is made of gelled alginate of various viscosity and guluronic/mannuronic acid residue ratios. In a specific embodiment, the inner housing is made of, primarily, low viscosity, high guluronic acid alginate (LVG: 73% guluronic content) alginate. In an alternative embodiment, the inner housing is made of, primarily, low viscosity, high guluronic acid alginate having guluronic acid concentration of about 30% to 85%, about 10% to 85% or guluronic concentrations of more than 50%, 55%, 60%, 65%, 70%, 75%, or 80%. The term “alginate,” as used herein, include any compound consisting of (1-4) linked beta-D-manuronic acid monomers and x-L-guluronic acid monomers.

The inner housing contains alginate beads or other materials in which cells can be encapsulated (e.g., agar, collagen, hyaluronic acid hydrogels, etc.). Within such alginate beads (or beads of other materials), cells of interest are entrapped or encapsulated. In one embodiment, cells are entrapped or encapsulated in high mannuronic acid content alginate beads, which are soft gels capable of maintaining cell viability in many cell lines. Because expansion of the cells beyond the beads is not desired, for certain cell lines, the outer alginate can be comprised of high guluronic acid content alginate, of varying viscosity, making a stiffer alginate that inhibits expansion. In a specific embodiment, the alginate beads comprise low viscosity, high guluronic acid alginate (LVG: 73% guluronic content) alginate and are encased within the inner housing. In an alternative embodiment, alginate beads are made of, primarily, low viscosity, alginate having guluronic acid concentrations of about less than 10%, about 10%-40%, about 10%-35%, about 10%-30%, about 10%-25%, about 10%-20%, about 10%-15%, about 30-50%, about 30-80%, about 50-80% or about 65%-80%.

The size of the alginate beads may be optimized by those skilled in the art to achieve optimal therapeutic effects, depending on various parameters, such as for example, the cell type, and the amount of cells housed, the site of implantation, the host species, and the material of the inner housing. Additionally, the beads may be of uniform or different sizes. In an embodiment, the diameter of the alginate beads is about 400 microns, to ensure oxygenation to the central-most cells. In certain embodiments, the diameter of the alginate beads is about 10 microns, 20 microns, 30 microns, 40 microns, 50 microns, 60 microns, 70 microns, 80 microns, 90 microns, 100 microns, 150 microns, 200 microns, 250 microns, 300 microns, 350 microns, 400 microns, 450 microns, 500 microns, 550 microns, 600 microns, 650 microns, 700 microns, 750 microns, 800 microns, 850 microns, 900 microns, 950 microns, or 1000 microns. In alternative embodiments, the diameter of the alginate beads is about 10 microns-1000 microns, 50 microns-950 microns, 100 microns-900 microns, 150 microns-850 microns, 200 microns-800 microns, 250 microns-750 microns, 300 microns-700 microns, 350 microns-650 microns, 400 microns-600 microns, or 450 microns-550 microns.

Various types of prokaryotic and eukaryotic cells may be used with the present implantable therapeutic device. Preferably, the encapsulated cells secrete one or more therapeutically useful substances, including but not limited to, hormones, growth factors, trophic factors, neurotransmitters, lymphokines, antibodies or other cell products that provide a therapeutic benefit to the device recipient. Examples of such therapeutic cell products include, but are not limited to, insulin, nerve growth factors, interleukins, parathyroid hormones, erythropoietins, albumins, and transferrins. Specific examples of therapeutically useful substances include, and are not limited to, IL-1, IL-2, IL-3, IL-4, IL-5, IL-6, IL-7, IL-8, IL-9, IL-10, IL-11, IL-15, Il-16, Il-18, IL-23, IL-24, erythropoietin, G-CSF, M-CSF, platelet derived growth factor (PDGF), MSF, FLT-3 ligand, EGF, fibroblast growth factor (FGF; e.g., aFGF (FGF-1), bFGF (FGF-2), FGF-3, FGF-4, FGF-5, FGF-6, or FGF-7), insulin-like growth factors (e.g., IGF-1, IGF-2); vascular endothelial growth factor (VEGF); interferons (e.g., IFN-γ, IFN-α, IFN-β); leukemia inhibitory factor (LIF); ciliary neurotrophic factor (CNTF); oncostatin M; stem cell factor (SCF); transforming growth factors (e.g., TGF-α, TGF-β1, TGF-β2, TGF-β3), or chemokines (such as, but not limited to, BCA-1/BLC-1, BRAK/Kec, CXCL16, CXCR3, ENA-78/LIX, Eotaxin-1, Eotaxin-2/MPIF-2, Exodus-2/SLC, Fractalkine/Neurotactin, GROalpha/MGSA, HCC-1, I-TAC, Lymphotactin/ATAC/SCM, MCP-1/MCAF, MCP-3, MCP-4, MDC/STCP-1, ABCD-1, MIP-1α, MIP-1β, MIP-2α/GROβ, MIP-3α/Exodus/LARC, MIP-3β/Exodus-3/ELC, MIP-4/PARC/DC-CK1, PF-4, RANTES, SDF1α, TARC, or TECK). In a specific embodiment, the encapsulated cells comprise endocrine-secreting cells. Polynucleotide sequences encoding therapeutically useful substances (sometimes referred to as “protein(s) of interest”) can be obtained from commercial databases such as EMBL, SWISSPROT, or the NCBI database.

Preferably cells of mammalian origin, or more preferably of human origin, are used with the present device. Useful cell types include, but are not limited to, insulin-secreting cells, lung cells, ovary cells, colon cells, kidney cells, prostate cells, pancreas cells, testes cells, cardiomyocytes, endothelial cells, epithelial cells, lymphocytes (T-cell and B cell), mast cells, eosinophils, vascular intimal cells, hepatocytes, leukocytes including mononuclear leukocytes, stem cells such as haemopoetic, neural, skin, lung, kidney, liver and myocyte stem cells, osteociasts, chondrocytes and other connective tissue cells, keratinoytes, melanocytes, liver cells, and adipocytes.

Particularly preferred cells are pancreatic beta cells or variants thereof. Suitable cells also include chondrocytes, osteocytes, osteoblasts, osteoclasts, mesenchymal stem cells, fibroblasts, muscle cells, hepatocytes, parenchymal cells, cells of intestinal origin, nerve cells, and skin cells, and may be provided as primary tissue explants, preparations of primary tissue explants, isolated cells, cell lines, transformed cell lines, and host cells. Suitable cells also include known research cells, including but not limited to, Jurkat T cells, NIH3T3 cells, and CHO cells.

In addition, the present therapeutic device may contain cells which have been genetically altered to contain at least one additional nucleic acid sequence related to the expression of a therapeutic substance. These genetically altered cells are distinguishable from naturally occurring cells, which do not contain the additional nucleic acid sequence. The additional nucleic acid sequences may be heterologous or homologous to the cells expressing the therapeutic substance. In addition, the additional nucleic acid sequences may encode for the therapeutic substance itself and/or comprise non-coding sequences, e.g. regulatory or antisense sequences which modify the expression of endogenous genes. Among the forms of nucleic acid sequences which may be useful for having been inserted into the genetically altered cells are intronless coding sequences (i.e. cDNA), copies of genomic genes, and regulatory sequences. The additional nucleic acid sequences may be comprised of sequences obtained from other cells, viruses, or synthetic sequences.

In an embodiment, the present therapeutic device contains naturally occurring and/or recombinant insulin-secreting cells, useful for treating diabetes. The insulin secreting cells may be of mammalian, more specifically, of human origin. In an embodiment, the insulin-secreting cells are pancreatic beta cells, for example, human pancreatic beta cells or murine (βTC-tet cells. In addition, the insulin secreting cells may be genetically engineered, and may further express additional islet proteins such as glucagon, somatosatin, and other pancreatic polypeptides for regulating blood glucose levels.

The number of encapsulated cells in each alginate bead can be adapted by those skilled in the art depending on parameters, including but not limited to, the cell type, the therapeutic substances produced by the cells, the desired treatment effects, the severity of the condition, the size of the alginate beads, and the desired site of implantation. In certain embodiments, the number of encapsulated cells in each alginate bead is about 1×10²-9×10⁹, 3×10²-3×10⁹, 5×10²-5×10⁹, 7×10²-7×10⁹, 9×10²-1×10⁹, 1×10³-9×10⁸, 3×10³-5×10⁸, 5×10³-5×10⁸, 7×10³-7×10⁷, 9×10³-1×10⁷, 1×10⁴-9×10⁶, 3×10⁴-5×10⁶, 5×10⁴-5×10⁶, 7×10⁴-7×10⁶, or 9×10⁴-1×10⁵.

The present therapeutic device may further comprise additional therapeutic substances useful for treating or amelioration of a disease or condition. Useful therapeutic substances include, but are not limited to, anti-diabetic agents such as agents that regulate the uptake of glucose, biguanides, sulfonylureas, insulin or insulin mimetics, alpha-glucosidase inhibitors. and anti-inflammatory agents.

The present implantable therapeutic device may be implanted to a variety of bodily location/site in the recipient that allows the device to release therapeutic substances, achieve the desired therapeutic effects, and/or provide the necessary nutrients to maintain the viability of cells within the device. Suitable sites for implanting the present device include, but are not limited to, the pancreas, the abdominal cavity, the peritoneal cavity, cerebral ventricles, and inside blood vessels. Further, the present device may be located subcutaneously or intramuscularly.

The therapeutic effects of the present therapeutic device may be monitored by various imaging techniques, such as for example, nuclear magnetic resonance spectroscopy (NMR), nuclear magnetic resonance imaging (NMRI), computed tomography scan (CT), ultrasound, radiography, gamma cameras, positron emission tomography (PET), endoscopy, thermography, medical photography, and microscopy.

Thus, the present implantable therapeutic device may further comprise an additional device such as one or more NMR coil, allowing for non-invasive observation and monitoring the host responses upon implantation. In an embodiment, the present device comprises radiofrequency coils that can be tuned to precise frequencies to allow for NMR signals to be obtained from only the entrapped cells. For example, viable cell numbers can be determined through NMR spectroscopy, by analysis of the total choline peak within the ¹H NMR spectra. NMR coils may be external surface coils. Further, to reduce signal-to-noise ratio (SNR) and signal contamination from intervening tissues (e.g., skin, muscle) and enhance detection of cells within the device, improved NMR sensitive coils can be manufactured that comprise an internal coil inductively coupled to an external coil. Such NMR sensitive coils not only produce NMR spectroscopic and imaging data with a SNR enhancement of about 2 fold, but also generate minimal unwanted signals from tissues outside of the present device, thereby allowing localized study to only the cells and materials contained within the implant core.

In an embodiment, the additional devices, such as NMR coils, are embedded in the outer housing. As a result, the outer housing is made of a material capable of electrically insulating the coil from the encapsulated cells and the host.

### Methods of Manufacture

Another aspect of this invention provides a method for manufacturing the present implantable therapeutic device, comprising:

a) providing liquid polydimethylsiloxane (PDMS);

b) forming the liquid polydimethylsiloxane (PDMS) into an outer housing wherein at least part of the outer housing is an open hollow cavity;

c) providing a liquid alginate having a guluronic acid content of about 10%-40%;

d) dispersing cells of interest within the liquid alginate to form an alginate-cell suspension;

e) forming gelled alginate beads encapsulating cells of interest from the alginate-cell suspension by gelling with a divalent cation (such as, but not limited to Mg++, Ca++, Ba++);

f) mixing the alginate beads with the liquid alginate having a guluronic acid content of about 50%-80%; and

g) filling the outer housing with the alginate beads/alginate mixture and spinning the outer housing until the alginate gels by gelling with a divalent cation (such as, but not limited to Mg++, Ca++, Ba++) to form an inner housing;

wherein the inner housing is situated inside the open hollow cavity of the outer housing, and wherein the alginate beads encapsulating cells of interest are enclosed inside the inner housing.

Another aspect of this invention provides a method for manufacturing the present implantable therapeutic device, comprising:

a) providing a material that can be formed into an outer housing wherein at least part of the outer housing is an open hollow cavity;

b) providing a liquid alginate having guluronic acid content of about 10%-40%;

c) dispersing cells of interest within the liquid alginate to form an alginate-cell suspension;

d) forming alginate beads encapsulating cells of interest from the alginate-cell suspension by gelling with a divalent cation of variable concentration (such as, but not limited to Mg++, Ca++, Ba++);

e) mixing the alginate beads with a liquid alginate having a guluronic acid content of about 50%-80% or about 10%-85%; and

f) filling the outer housing with the alginate bead/alginate mixture and spinning the outer housing while the alginate gels by gelling with a divalent cation (such as, but not limited to Mg++, Ca++, Ba++) of variable concentration to form an inner housing;

wherein the inner housing is situated inside the open hollow cavity of the outer housing, and wherein the alginate beads encapsulating cells of interest are enclosed inside the inner housing.

In either embodiment, the use of rotational motion such as a centrifugal motion counteracts the natural shrinkage of the alginate while it gels, thus trapping the alginate core into the outer housing. Optionally, and additionally, one or more imaging devices such as NMR coils may be immersed in the liquid PDMS or other material used to form an outer housing, forming the outer housing comprising NMR coils. Further, the alginate mixture has a guluronic acid content of about less than 10%, about 10%-80%, about 10%-40%, about 10%-35%, about 10%-30%, about 10%-25%, about 10%-20%, about 10%-15%, about 30-50%, about 10-85%, about 30-80%, about 50-80% or about 65%-80%. As discussed above, the outer housing may, optionally, be coated with a biologically inert material, such as PDMS.

### Benefits of the Implantable Therapeutic Device

The present implantable therapeutic device is useful for treating or ameliorating diabetes or diabetic conditions of a subject, including but not limited to, type-I diabetes mellitus, hyperglycemia, impaired glucose tolerance, insulin deficiency, elevated glucose levels, and insulin resistance. Further, the present device is useful for treating or ameliorating a symptom or condition associated with diabetes or insulin resistance, including but not limited to, obesity, lipid disorders, dyslipidemia, hyperlipidemia, hypertriglyceridemia, hypercholesterolemia, stroke, retinopathy, diabetic ketoacidosis, diabetic neuropathy, cardiovascular diseases, kidney diseases, diabetic nephropathy, low HDL levels, high LDL levels, atherosclerosis, vascular restenosis, irritable bowel syndrome, inflammatory conditions, diabetes-related skin conditions such as dermopathy, necrobiosis lipodica diabeticorum, diabetic blisters, gum diseases, pancreatitis, neurodegenerative disease, and other diabetes-associated disorders or complications.

The implantable therapeutic devices disclosed herein can also be used for the treatment or amelioration of diseases associated with the underproduction of various endocrine molecules. Non-limiting examples of endocrine molecules that can be delivered using the devices disclosed herein include: norepinephrine; epinephrine; dopamine; thyroid hormones, such as 3,5,3′-triiodothyronine (T3) and 3,5,3′,5′-tetraiodothyronine (thyroxine, T4); follicle stimulating hormone (FSH), thyroid stimulating hormone (TSH), endorphins, luteinizing hormone (LH), prolactin, growth hormone, parathyroid hormone, leptin, ghrelin, cortisol, corticosterone, testosterone, melanocyte stimulating hormone, adrenocorticotropic hormone (ACTH), oxytocin, antidiuretic hormone, aldosterone, dehydroepiandrosterone (DHEA), DHEA sulfate (DHEA-S), or other glucocorticoids, mineralocorticoids, androgens, estrogens and/or progestagens. Such endocrine molecules can be secreted from isolated tissue cells that secrete these molecules (e.g., cells from the thyroid, parathyroid, adrenal or pituary tissues) or by cells genetically engineered to secrete such endocrine molecules.

As demonstrated in the Examples, pancreatic islet cells can be successfully delivered via the present implantable therapeutic device into a subject in need of such treatment. Upon implantation, these pancreatic islet cells secrete insulin in response to glucose levels. Thus, the present device can provide useful treatment effects such as reversing or reducing hyperglycemia, sustaining the diabetic subject for a prolonged period of time, and/or inducing beta cell regeneration. In an embodiment, the present device is used as an artificial pancreas.

Further, although the treatment of diabetes is specifically exemplified herein, the present device can be used to deliver a variety of cells, tissues, and/or therapeutic substances into a subject in need of such treatment. For example, cells expressing and/or secreting one or more therapeutic substances, such as hormone-secreting cells, can be delivered into a desired bodily location. Further, various tissues, such as thyroid, parathyroid and adrenal tissues, can be entrapped or encapsulated in alginate beads and implanted into a subject. Particularly, the present device is useful for treating or ameliorating diseases or disorders associated with abnormal cellular secretion.

In addition, the present therapeutic device can be used as a research tool in animal models for studying pathogenesis and treatment of diseases.

For example, a major challenge in diabetic research involves the lack of ability to maintain the diabetic animal for a prolonged period of time. Diabetic animal, especially small rodents, quickly succumb to hyperglycemic effects in a matter of days and usually cannot survive long enough for ongoing studies such as identifying the effects of hyperglycemia on targeted organs. Currently, diabetic animals are sustained by constant insulin injections or the insertion of passive insulin pumps. However, insulin injections or insulin pumps are usually not well-standardized and unable maintain glucose levels exquisitely. Further, they do not contain mouse insulin, thereby unsuitable for use in mouse models.

Advantageously, the present device allows for exquisite regulation of blood glucose levels, and thereby capable of sustaining hyperglycemic laboratory animals for a prolonged period of time. Thus, animals implanted with the present device represent a superior model system for studying the pathogenesis of diabetes mellitus, in particular diabetes mellitus type 1. For example, diseased laboratory animals implanted with the present device can sustain for a longer period of time; therefore, the present device is useful for long-term studying the onset, development, and progression of diabetes, for studying glucose uptake and regulation, for monitoring pancreatic cell mass, and studying hormone such as insulin secretion, and for studying the effects of hyperglycemia on targeted organs and diabetes-associated complications.

In addition, animals implanted with the present device represent a superior model system for studying the therapy or treatment of diabetes mellitus, in particular diabetes mellitus type 1, such as for example, for identifying means and methods suitable for the therapy or treatment of diabetes mellitus, in particular diabetes mellitus type 1, for drug screening and developing regenerative medicines such as regeneration of pancreatic beta cells, and for studying host immune responses.

Further, although the study of diabetes is specifically exemplified herein, the present device can be implanted to sustain a variety of laboratory animal for a prolonged period time for studying a variety of diseases. For example, cells expressing and/or secreting one or more peptides, hormones, lipids or other substances of interest can be introduced into an animal via implantation, for studying the pathogenesis and/or treatment of the diseases or disorders. Additionally, one or more imaging devices may be included in the present device, allowing for non-invasive observation and monitoring of the host responses.

The devices disclosed herein can also be used to augment or increase the levels of a given hormone, cytokine or other endocrine molecule. In such aspects of the invention, hormone, cytokine or other endocrine molecule secreting cells (transformed to express the hormone. cytokine or other endocrine molecule or non-transformed cells that naturally express the hormone, cytokine or other endocrine molecule) are encapsulated within the devices disclosed herein. These devices are then implanted into a subject (e.g., an animal or a human) thereby facilitating the augmentation of the hormone, cytokine or other endocrine molecule within the subject.

All patents, patent applications, provisional applications, and publications referred to or cited herein are incorporated by reference in their entirety, including all figures and tables, to the extent they are not inconsistent with the explicit teachings of this specification.

Following are examples which illustrate procedures for practicing the invention. These examples should not be construed as limiting. All percentages are by weight and all solvent mixture proportions are by volume unless otherwise noted.

### Example 1

### Structure of the Implantable Therapeutic Device

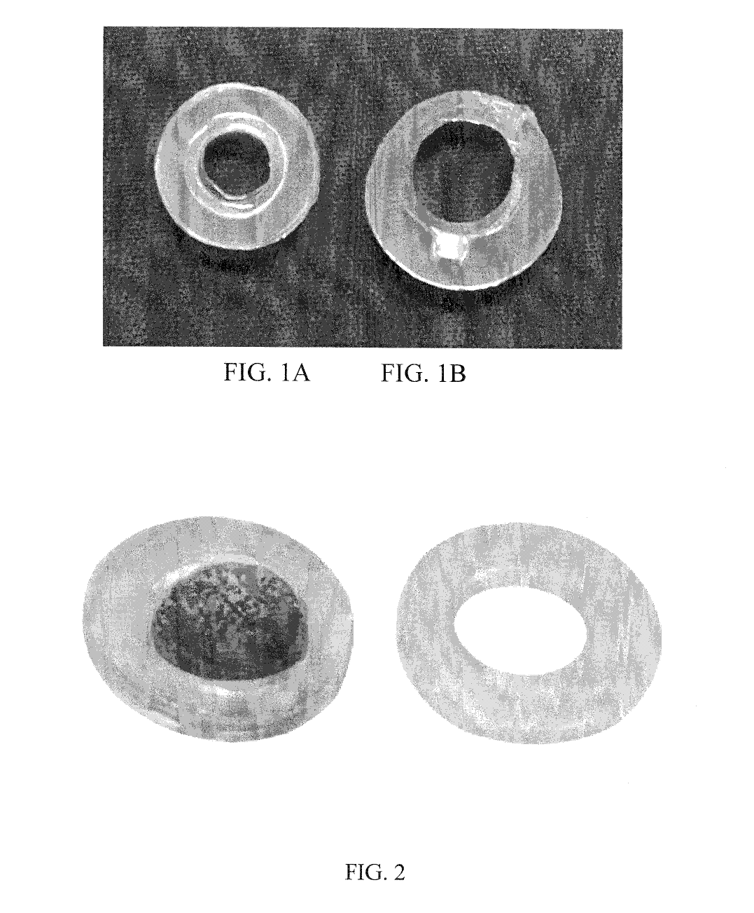

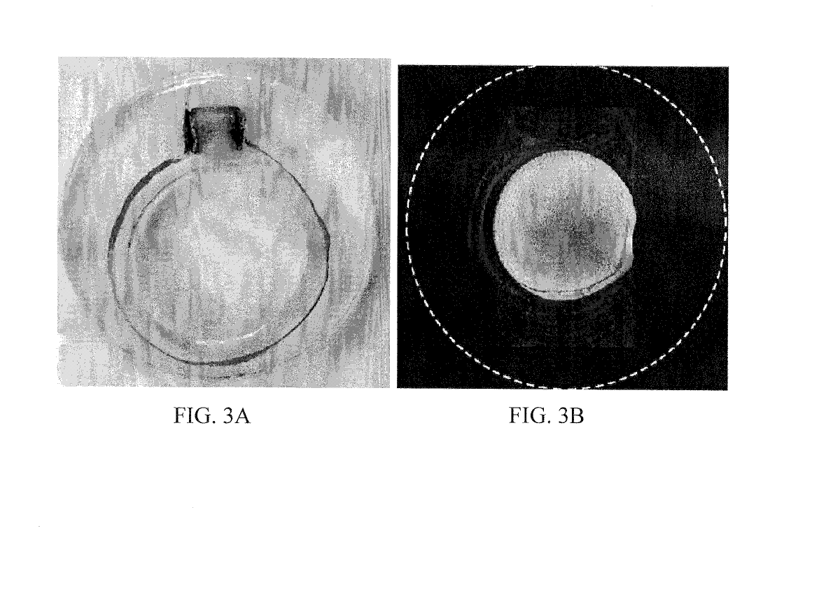

This Example provides an embodiment of the implantable therapeutic device. The device, with the top-most PDMS ring removed, is shown in FIGS. 1 and 2. There are three PDMS rings: one on the top, one on the bottom (˜1 mm thick, ˜14 mm outer diameter, ˜9.5 mm inner diameter), and one in the center (˜2 mm thick, ˜14 mm outer diameter, ˜12 mm inner diameter). Insulin-secreting cells are entrapped in alginate beads (as shown in blue in FIG. 2 for visual effect), and these beads are entrapped in alginate that fills the core of the construct. The ledge that keeps the alginate core within the PDMS construct is visible.

### Example 2

### Methods of Manufacturing the Implantable Therapeutic Device

This Example provides an exemplified method for manufacturing of the implantable cell encapsulation device. First, biomedical-grade polydimethylsiloxane (PDMS: Factor II, Lakeside Ariz.) is formed into uniformly thick sheets of 1 and 2 mm. If NMR coils are to be inserted, they are placed into the thicker layer while the PDMS is allowed to cure. A cork borer is used to create the desired inner and outer diameters. These layers are attached to each other with liquid PDMS. Because PDMS is a cross-linked polymer, the PDMS layers bond to each other. These PDMS rings create an outer housing into which the alginate inner housing can be situated. Because alginate does not stick to PDMS, the center slab inner diameter (—11-12 mm) is slightly larger than the outer slabs inner diameter (−9.5 mm). This creates a center ledge, which traps the gelled alginate into the outer housing. The PDMS is acid washed for 1 hour in 1M HCl to remove proteins, rinsed in deionized water, and autoclaved to sterilize.

Two types of alginate are used: (i) low viscosity, high mannuronic acid alginate (LVM: 38% guluronic content) to entrap the cells; and (ii) low viscosity, high guluronic acid alginate (LVG: 73% guluronic content) to solidify into the inner housing of the device (both supplied by NovaMatrix (Oslo. Norway)). Then, βTC-tet cells (provided by Shimon Efrat) are entrapped into 2% w/v sodium alginate beads by procedures known in the art (See Lim and Sun, which are incorporated by reference in their entireties; Lim 1980; Sun et al., 1980; Simpson et al., 2005; Simpson et al., 2003; Simpson et al,. 2006a; Simpson et al., 2004; Simpson et al. 2006b). In this embodiment, the cell density at encapsulation is 3.5×10⁷ cells/ml alginate. Cells are cultured and mixed with 2% LVM alginate, and then an electrostatic bead generator (Nisco, Zurich, Switzerland) is used to create beads with 400 micron in diameter by crosslinking with 0.28% BaCl₂. These beads are washed three times in 0.28% BaCl₂, three times in 0.14% BaCl₂, and three times in phosphate buffered saline. The beads are mixed (1 part to 2 or 3 parts) with liquid 2% LVG alginate.

In this Example, the mouse beta-cell line βTC-tet is used so that syngeneic and allogeneic mouse models can be employed for studying host immune responses upon implantation, evaluating the materials of the present device (Simpson et al., 2003; Grant et al., 2005), studying cellular function (Simpson et al., 2006a), and determining viability of the entrapped cells (Simpson et al., 2004; Simpson et al., 2006b; Grant et al., 2005; Oca-Cossio et al., 2005; Simpson et al., 2005). Additionally, the amount of βTC-tet cells in alginate beads can be easily regulated due to the inclusion of the bacterial tet-operon. Exposure to tetracycline, an antibacterial agent, inhibits βTC-tet cellular division (Efrat et al., 1995; Fleischer et al., 1998). Further, entrapped βTC-tet cells can maintain glucose-responsiveness (i.e., cells secrete insulin in a glucose-dependent manner) over long periods (Simpson et al., 2005).

Because alginate will contract during the gelling process, it is less desirable to just pour the alginate bead solution into the construct and gel it, as it may cause the alginate core diameter to shrink, and thus come out of the construct. To solve this problem, we developed a novel approach using centrifugal force to keep the alginate into the construct ledge while the alginate is gelling. Specifically, circular stir bars are taped to the bottom of high wall 60 mm-diameter Petri dishes. These Petri dishes are placed onto a stir plate. A glass plate larger than the Petri dish is placed between the Petri dish and the stir plate, to allow for reduced friction and allow spinning. Sterile construct formers made from PDMS that fit precisely into a sterile 60 mm Petri dish, and contain a ˜21 mm-diameter center hole, are placed into the Petri dishes. Advantageously, this not only keeps the construct steady during rotation, but also reduces the volume of gelling agent needed to cover the construct during gellation. A piece of sterile filter paper, wetted with 1.1% CaCl₂, is placed underneath the construct holder to facilitate the initiation of the gelling process and keep the alginate from leaking underneath. The sterile PDMS constructs are placed in the center, and the alginate/bead mixture is added. Another piece of sterile filter paper, wetted with 1.1% CaCl₂, is placed on top of the liquid alginate, which also facilitates the initiation of the gelling process, and prevents the gelling solution to be added from displacing the liquid alginate in the construct while allows the gelling ions to pass through. The stir plate is spun at 180-240 rpm, and 1.1% CaCl₂ is added to each dish to cover the construct. When gelling is completed in 20 minutes, the constructs are removed, placed into small wells, rinsed and stored in a buffered saline solution, ready for use as implants.

The protocol for manufacturing the PDMS/alginate constructs is illustrated in further details as follows. Materials include: 0.55% BaCl₂, LVM alginate beads (generally ˜400 micron diameter), 2% LVG alginate, sterile PDMS constructs, sterile filter paper disks (˜1″ diameter, and ˜15 mm diameter), sterile PDMS formers (6) which are donut shaped items which fill the 60 mm Petri dish and has a hole (˜21 mm diameter) that allows the construct to easily fit. This device reduces the volume needed to gel the alginate and keeps the construct in the center of rotation during the gellation process, sterile forceps (at least 2), glass dishes (up to 6), circular magnetic stir-bars (6), 6-place magnetic stir plate, pipets (25 ml), sterile pipets, insulin syringe (0.5 ml) and needle (29 ga), 1.1% CaCl₂, 0.55% CaCl₂, Hank's Balanced Buffered Salts (with penicillin/streptomycin; dexamethasone), 50 ml centrifuge tube, ‘deep dish’ 60 mm Petri dishes (6), and lab tape.

Exemplary steps of the set-up procedure are illustrated as follows:

1. Place a magnetic stir plate in a tissue-culture hood.

2. Arrange glass plates onto the stir plate.

3. Tape circular magnetic stir-bars onto sterile high wall 60 mm Petri dishes and ensure the Petri dishes can spin.

4. Wet sterile paper disks in 1.1% CaCl₂ and place the wetted paper disks onto the center of each Petri dish.

5. Using forceps, carefully place PDMS formers into Petri dishes, snug to bottom.

6. Place PDMS constructs into the center of PDMS formers.

7. Ensure that each of the Petri dishes can spin well.

Essential steps of manufacturing the construct are illustrated as follows:

1. Take settled beads (rinsed in DPBS) and place the beads into the centrifuge tube.

2. Calculate the volume of settled beads (=volume×˜0.64).

3. Remove all liquid from the centrifuge tube with syringe and small needle.

4. Add 2 ml 2% LVG alginate for each 1 ml beads and mix well: it should have a consistency of “malt-o-meal.”

5. With pipet, add bead/alginate mix to each construct.

6. Spin rapidly to force alginate to sides. Remove air bubbles with syringe.

7. Add more alginate/bead solution if necessary.

8. Add small diameter paper (wetted with 1.1% CaCl₂) to the top.

9. Carefully add ˜5 ml 1.1% CaCl₂ to each dish so that alginate is not disturbed.

10. Spin the dish for about 20 minutes until alginate gels completely. During spinning, add CaCl₂ continuously until the construct in the plate is covered by the solution.

11. After centrifugation, carefully lift the construct to expose the underside to the solution and remove the construct with new forceps and place into a 12-well plate.

12. Rinse the constructed device with 0.55% CaCl₂, remove the solution.

13. Add 2 ml Hank's buffered saline (with dexamethasone, pen/strep).

The resulting implantable therapeutic device is ready for implantation into a subject.

### Example 3

### Therapeutic Effects of the Implantable Therapeutic Device

Mouse models of diabetes offer fertile ground to study β-cell regeneration, effects of hyperglycemia on organ systems, and cures for diabetes. In the course of diabetic research, rendering an animal diabetic is relatively easy; however, maintaining hyper- or normoglycemic states for extended periods remains challenging. To solve this problem, the present invention provides implantable therapeutic devices comprised of insulin-secreting cells entrapped in biocompatible materials. When implanted, cells in the device sense the recipient's glucose levels and secrete insulin appropriately, thereby regulating blood sugar levels.

This Example illustrates the in vivo results of the present device upon implantation. In a first set of experiments, four C3H/HeN female mice were rendered diabetic (defined herein as having fasting blood glucoses >300 mg/kg for 3 days) through a single intravenous (i.v.) injection of alloxan (62.5 mg/kg dose). Once the mice became diabetic, the present device was surgically implanted into the peritoneal cavity above the intestine. The device contains βTC-tet cells entrapped in the alginate beads (3.5×10⁷ cells/ml 2% LVM alginate). The beads are contained within the inner housing of the device with 2% LVG alginate.

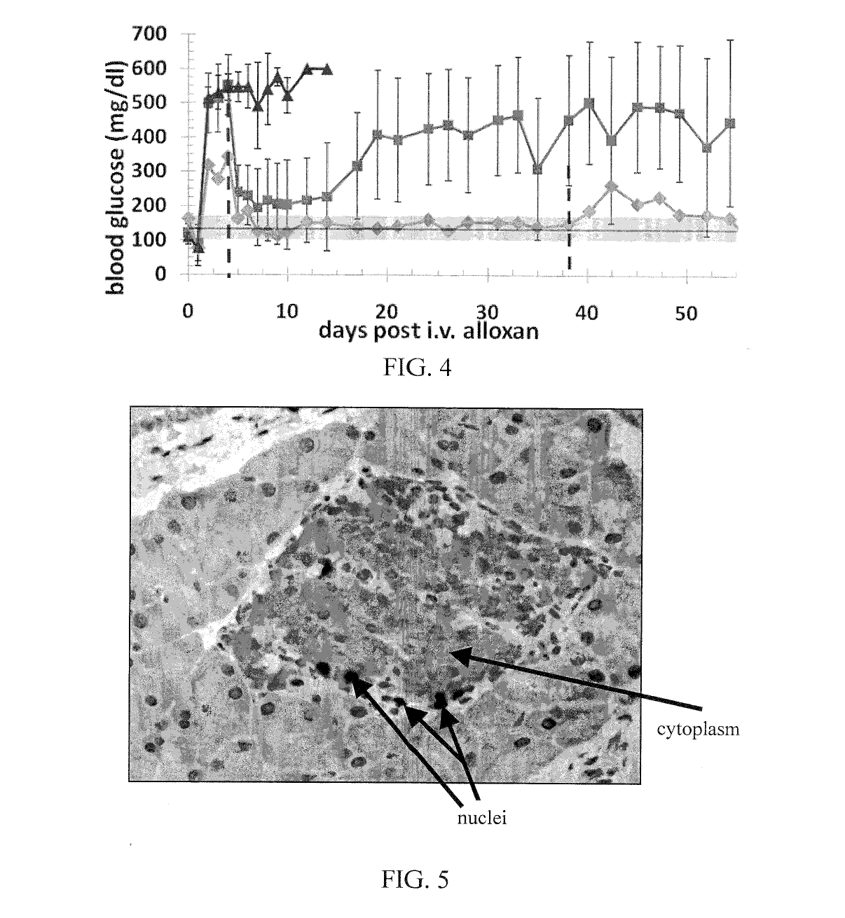

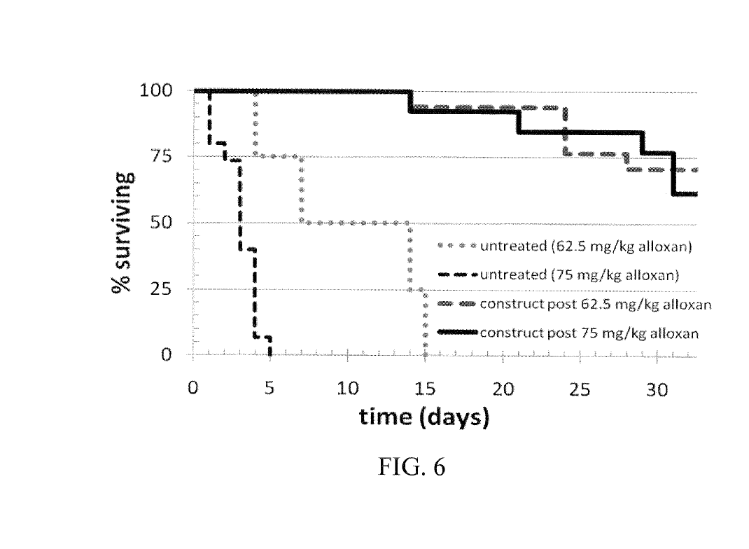

In each trial, one diabetic mouse was effectively cured by the implant. The cured mice exhibits normal average blood sugar levels (plus/minus one standard deviation are shown in the grey band in FIG. 4). Specifically, in the first trial, after 38 days, the implant was removed. Although the blood sugar level of the cured mouse reverts to hyperglycemic immediately after the removal of the implant, it slowly drifts back to normal. Along with histologic staining which showed division of insulin-containing cells (see FIG. 5), this change demonstrates that implantation of the present device maintains animal health long enough to successfully allow for in vivo beta cell regeneration. FIG. 6 illustrates the survival of animals treated with the devices disclosed in this application. As will be noted from the Figure, animals implanted with βTC-tet cells entrapped in alginate beads survive much longer than animals that were untreated.

For any diabetic mice that are not completely cured by the implant, the present device nonetheless provides substantial treatment effects by keeping the mice alive at a hyperglycemic level far longer than if they had not received implantation. Therefore, the present device can serve as a research tool for studying the effects of hyperglycemia on target organs (e.g., retina, kidney, heart, etc.). By using a subefficacious cell number, such an effective approach can be taken.

In a second set of experiments, diabetic female mice (C3H/HeN) are implanted with implants containing βTCtet insulinoma cells (˜4×10⁶ cells).

The results indicated that mice exhibit a significant characteristic drop in fasting blood glucose levels one day post-alloxan injection (p<0.01), presumably due to beta cell death and insulin dumping. Once mice become diabetic (defined herein as having fasting blood glucoses >300 mg/kg for 3 days), the present device is crafted and implanted intraperitoneally. At time of implantation, fasting blood glucose levels are significantly higher than normal fasting values (p=0.000014).

One day after implant, fasting blood sugar levels drop significantly (p=0.001, all; p<0.0005, responders) and gradually return toward normal. One mouse survived to day 28 and the device was removed on day 38. The removal of the device resulted in a first rise in fasting blood glucose, followed by a subsequent return to normal levels.

On day 55, the cured mouse was euthanized and its pancreas is removed for testing beta cell regeneration. Recent studies reveal that endogenous recovery of beta cells occurs if sufficient time passes while the animal is sustained and there is no immune response against the beta cells (Yin et al., 2006). The results (Ki67 and insulin positive in islets) revealed that implantation of the present therapeutic device induces beta cell regeneration.

In the third set of experiments, female C3H/HeN mice (n=8) are rendered diabetic (defined herein as having fasting blood glucoses >300 mg/kg for 3 days) by one intravenous (i.v.) injection of alloxan (62.5 mg/kg). Once mice become diabetic, the present device containing βTC-tet insulinoma cells was crafted and implanted into the peritoneal cavity above the intestine. Fasting blood sugar levels are monitored following implantation. Mice with sustained blood glucose >600, or >15% mass loss were euthanized (B,C), and the device, eyes, liver, pancreas, heart and kidneys removed for histology.

The results revealed that alloxan usually (⅞) induces low blood sugar 24 h post administration, due to β-cell death and insulin dumping. Thereafter, hyperglycemia occurs, until corrected by implantation (on day 4). All mice exhibited reduced fasting blood sugar levels immediately post-implantation and five mice (A,D,F,G,H) exhibited near normal blood sugar levels, which demonstrate that the present device provides effective treatment effects for diabetes. In addition, three mice maintained a hyperglycemic state for an extended time. On day 19 post alloxan, Mouse B exhibited a drop in blood sugar levels, indicating insulin dumping due to cell death within the device. After euthanasia, minimal fibrotic response to the implant (e.g., fibrotic overgrowth) was observed.

In conclusion, this Example demonstrates that the present device is biocompatible, effective in regulating the blood glucose levels for an extended period, and useful for treating for diabetes. Additionally, this Example demonstrates that the present device can serve as a useful research tool for diabetes researches. It enables a researcher to determine if β-cell regeneration is occurring in the damaged pancreata of sustained animals, and observe the effects of extended hyperglycemia on target organs.

### Example 4

### Effects of the Implantable Therapeutic Device on an Allogeneic Spontaneous Animal Model for Type 1 Diabetes (T1d)

This Example aims to further examine the ability of the present therapeutic device for treating diabetes in an animal model, studying the optimization of the cell number of contained in the device to enhance treatment effects, determining the longevity of the implant, and studying the correlation between cell viability (determined non-invasively through NMR methods) with the function of the implant.

Specifically, devices are manufactured, filled with alginate beads containing βTC-tet cells, and implanted intraperitoneally into alloxan-induced (62.5 mg/Kg) diabetic female C3H/HeN mice (n=18-20). These mice are MHC matched to this cell line (H-2k). The mice are divided into two groups: the first group consists of diabetic mice receiving cellular devices (n=12-15, experimental); and the second group consists of diabetic mice receiving acellular devices (n=6, negative control). Fasting blood sugar levels are measured at least 3 times weekly. One day post-implantation, and every two weeks thereafter, NMR ¹H spectroscopy at 11T is performed on 6 experimental animals to non-invasively estimate the number of viable cells within the device based on the total choline peak in the spectrum (Stabler et al., 2005a; Stabler et al., 2005b). NMR imaging (diffusion-weighted and T2-weighted) is performed to study the structural changes to the device over time, as well as identify immune cell infiltration or fibrosis. At the conclusion of the study, devices are removed for histological examination; e.g., sectioned and stained with hematoxylin/eosin (live/dead), IHC (insulin), and the pancreas removed, fixed and examined for evidence of beta cell re-growth (insulin & Ki67 staining).

The results will show that animals receiving acellular devices will remain diabetic and rapidly succumb to diabetes. Animals receiving cellular devices will thrive and become normoglycemic. The results will also show that the glucose-responsiveness will not be lost (Simpson et al., 2005). Depending on the amount of insulin released over time, the number of cells contained in the device can be adjusted by altering the cellular density. Data from conducted experiments do not suggest that overgrowth of the implanted cells is an issue. However, cellular pretreatment with tetracycline (TC) of cells to be encapsulated (controlling division for ˜80 days (Simpson et al., 2005) or TC ingestion through the water supply (Black et al., 2006) can be implemented if sustained hypoglycemic states are encountered. Further, NMR data will provide correlation between the function of the device with the viable cells contained within it, as well as identify structural changes to the device.

### Example 5

### Effects of the Implantable Therapeutic Device on an Allogeneic Spontaneous Animal Model for T1d

This Example aims to reveal that the implant system is translatable to humans (i.e., an allogeneic system). Although stem cell derived beta cells and autologous cell systems (Black et al., 2006) are critical approaches toward making ideal cells for inclusion in an implantable device, at present, β-cells or islets cannot always be immune matched to a recipient; therefore, allogeneic animal model studies are warranted. Furthermore, a spontaneous diabetes mouse model caused by the immune destruction of beta cells is more appropriate for modeling human type 1 diabetes (T1D).

As described in Example 4, the present device is manufactured, filled with alginate beads containing βTC-tet cells, and implanted intraperitoneally into diabetic female NOD mice (n=12-15) (Atkinson et al., 1999). This cell and animal line model is appropriate for studying allogeneic systems, as it has been shown by Cattan et al. that βTC-tet cells (H-2k) were rejected when injected into NOD (H-2g7) mice (Cattan et al., 2003). To counter this adverse immune response, we have experience for inducing immune tolerance in NOD mice with anti-thymocyte globulin (ATG) (Simon et al., 2008; Parker et al., 2009), and thus, can model a potential future clinical situation where immune suppression is needed for acceptance of a mismatched implant. The recurrence of autoimmunity will also be addressed by this treatment.

To induce tolerance, recipient NOD animals are injected twice i.p. with 500 micrograms ATG: the first time when a diabetic state has been established, and the second time 72 hours later (Simon et al., 2008; Parker et al., 2009). Fasting blood sugar levels are measured at least 3 times weekly. One day post-implantation, and every 2 weeks thereafter, NMR ¹H spectroscopy at 11T is performed on 6 experimental animals to non-invasively estimate the number of viable cells within the device based on the total choline peak in the spectrum (Stabler et al., 2005a; Stabler et al., 2005b). At the conclusion of the study, the devices are removed for histological examination; e.g., sectioned and stained with hematoxylin/eosin (live/dead), IHC (insulin). The pancreas is removed, fixed and examined for beta cell regrowth (insulin & Ki67 staining).

The results will show that animals receiving cellular devices will thrive and become normoglycemic, demonstrating efficacy in an allogeneic model. ATG therapy should not induce a cytokine storm (Simon et al. 2008), although transient cytokine increases may occur. These changes will be monitored by a cytokine panel assay (MilliPlex), as described in Goudy et al. (2003). If rejection is an issue, MR imaging and spectroscopic studies will show evidence of an immune attack before normoglycemia is lost, evidencing that the NMR monitoring approach of the present invention can predict device failure.

### Example 6

### Effects of the Implantable Therapeutic Device on Pancreatic Insulin Secretion

This Example aims to reveal the significance of residual pancreatic function on the changes to blood sugar levels of diabetic mice following implantation of the bioartificial pancreas. Specifically, diabetic NOD mice are implanted with devices containing human islets (n=16-20). Animals are rendered immunotolerant by i.p. administration of ATG, and blood samples are tested for determining fasting blood glucose levels, insulin levels, cytokine presence, and C-peptide levels. Mouse and human C-peptide can be distinguished from each other; therefore, the device's contribution to the animal's recovery from diabetes can be better estimated. At the end of the study, each animal's device and pancreas are removed and tested as described in Examples 4 and 5.

The results will show that diabetes will be treated or ameliorated by the implantation of the device, and that the bulk of the insulin regulating the blood glucose arises from the human beta cells contained within the device. Given sufficient time rendered normoglycemic, it is possible that the native mouse beta cells may recover and regenerate. This Example will also demonstrate that combining NMR studies with bioluminescent studies on an animal with luciferase-expressing β-cells would allow non-invasive monitoring of both the device and the pancreas.

It should be understood that the examples and embodiments described herein are for illustrative purposes only and that various modifications or changes in light thereof will be suggested to persons skilled in the art and are to be included within the spirit and purview of this application. In addition, any elements or limitations of any invention or embodiment thereof disclosed herein can be combined with any and/or all other elements or limitations (individually or in any combination) or any other invention or embodiment thereof disclosed herein, and all such combinations are contemplated with the scope of the invention without limitation thereto.

### REFERENCES

King H., Aubert R, Herman W. Global burden of diabetes, 1995-2025. Prevalence, numerical estimates and projections. Diabetes Care, 1998; 21: 1414-1431.

Robertson R P. Seminars in medicine of the Beth Israel Hospital, Boston: Pancreatic and islet transplantation for diabetes--cures or curiosities? N Engl J Med, 1992; 327: 1861-1868.

Sutherland D E. Pancreas and islet cell transplantation: now and then. Transplant Proc, 1996; 28:2131-2133.

Shapiro A M, Lakey J R, Ryan E A, Korbutt G S, Toth E, Warnock G L, Kneteman N M, Rajotte R V. Islet transplantation in seven patients with type 1 diabetes mellitus using a glucocorticoid free immunosuppressive regimen. N Engl J Med, 2000; 343:230-238.

Lim F, Sun A M. Microencapsulated islets as bioartificial endocrine pancreas. Science, 1980; 210:908-910.

Sun A M, Parisius W, Macmorine H, Sefton M V, Stone R. An artificial pancreas containing cultured islets of Langerhans. Artif Organs, 1980; 4:275-278.

Simpson N E, Khokhlova N, Oca-Cossio J A, McFarlane S S, Simpson C P, Constantinidis I. Effects of growth regulation on conditionally-transformed alginate-entrapped insulin secreting cell lines in vitro. Biomaterials, 2005; 26:4633-4641.

Simpson N E, Grant S C, Blackband S J, Constantinidis I. NMR properties of alginate microbeads. Biomaterials, 2003; 24:4941-4948.

Simpson N E, Khokhlova N, Oca-Cossio J A, Constantinidis I. Insights into the role of anaplerosis in insulin secretion: a ¹³C NMR study. Diabetologia, 2006a; 49:1338-1348.

Simpson N E, Stabler C L, Simpson C P, Sambanis A, Constantinidis I. The role of CaCl₂-guluronic acid interaction on alginate encapsulated βTC3 cells. Biomaterials, 2004; 25:2603-2610.

Simpson N E, Grant S C, Gustavsson L, Peltonen Y-M, Blackband S J, Constantinidis I. Biochemical consequences of alginate encapsulation: a NMR study of insulin-secreting cells. Biomaterials, 2006b; 27:2577-2586.

Grant S C, Celper S, Gauffin Holmberg I, Simpson N E, Blackband S J, Constantinidis I. Alginate assessment by NMR microscopy. J Mat Sci: Mat Med, 2005; 16:511-514.

Oca-Cossio J, Simpson N E, Han Z, Stacpoole P W, Constantinidis I. Effects of alginate encapsulation on mitochondrial activity. J Mat Sci: Mat Med, 2005; 16:521-524.

Efrat S, Fusco-DeMane D, Lemberg H, Emran O A, Wang X. Conditional transformation of a pancreatic β-cell line derived from transgenic mice expressing a tetracycline-regulated oncogene. PNAS, 1995; 92:3576-3580.

Fleischer, N., Chen, C., Surana, M., Leiser, M., Rossetti, L., Pralong, W., and Efrat, S. Functional analysis of a conditionally transformed pancreatic β-cell line. Diabetes, 1998; 47:1419-1425.

Volland N A, Mareci T H, Constantinidis I, Simpson N E. Development of an inductively-coupled MR coil system for imaging and spectroscopic analysis of an implantable bioartificial construct at 11.1T. Magn Res Med, 2010; in press.

Stabler C L, Long R C J, Constantinidis I, Sambanis A. Noninvasive measurement of viable cell number in tissue engineered constructs using 1H NMR spectroscopy. Tissue Engin, 2005a; 11:404-414.

Stabler C L, Long R C J, Constantinidis I, Sambanis A. In vivo noninvasive monitoring of viable cell number in tissue engineered constructs using 1H NMR spectroscopy. Cell Transplant, 2005b; 14:139-149.

Hoult D I, Tomanek B. Use of mutually inductive coupling in probe design. Concepts in Magn Res, 2002;15:262-285.

Silver X, Ni W X, Maercer E V, Beck B L, Bossart E L, Inglis B, Mareci T H. In vivo 1H magnetic resonance imaging and spectroscopy of the rat spinal cord using an inductively-coupled chronically implanted RF coil. Magn Res Med, 2001;46:1216-1222.

Wirth E D I, Mareci T H, Beck B L, Fitzsimmons J R, Reier P J. A comparison of an inductively coupled implanted coil with optimized surface coils for in vivo NMR imaging of the spinal cord. Magn Res Med, 1993;30:626-633.

Yin D, Tao J, Lee D D, Shen J, Hara M, Lopez J, Kuznetsov A, Philipson L H, Chong A S. Recovery of islet beta-cell function in streptozotocin-induced diabetic mice: an indirect role for the spleen. Diabetes, 2006; 55:3256-3263.

Black S P, Constantinidis I, Cui H, Tucker-Burden C, Weber C J, Safley S A. Immune responses to an encapsulated allogeneic islet beta-cell line in diabetic NOD mice. BBRC, 2006; 340:236-243.

Bara H, Sambanis A. Development and characterization of a tissue engineered pancreatic substitute based on recombinant intestinal endocrine L-cells. Biotech Bioengin. 2009; 104:824-834.

Atkinson M A, Leiter E H. The NOD mouse model of type 1 diabetes: as good as it gets? Nat Med, 1999; 5:601-604.

Cattan P, Rottembourg D, Cottet S, Tardivel I, Dupreaz P, Thorens B, Boitard C, Carel J C. Destruction of conditional insulinoma cell lines in NOD mice: a role for autoimmunity. Diabetologia, 2003; 46:504-510.

Simon G, Parker M, Ramiya V, Wasserfall C, Huang Y, et al. Murine antithymocyte globulin therapy alters disease progression in NOD mice by a time-dependent induction of immune regulation. Diabetes, 2008; 57:405-414.

Parker M J, Xue S, Alexander J J, Wasserfall C H, Campbell-Thompson M L, Battaglia M, Gregori S, Mathews C E, Song S, Troutt M, Eisenbeis S, Williams J, Schatz D A, Haller M J, Atkinson M A. Immune depletion with cellular mobilization imparts immunoregulation and reverses autoimmune diabetes in nonobese diabetic mice. Diabetes, 2009; 58:2277-2284.

Goudy K S, Burkhardt B R, Wasserfall C, Song S, Campbell-Thompson M C, et al. Systemic overexpression of IL-10 induces CD4+ CD25+ cell populations in vivo and ameliorates type 1 diabetes in nonobese diabetic mice in a dose-dependent fashion. J Immuno, 2003; 171:2270-2278.

1-33. (canceled)

34. An implantable therapeutic device, comprising:
a) an outer housing made of a formable material, wherein the outer housing comprises an open hollow cavity;
b) an inner housing situated inside the open hollow cavity of said outer housing, wherein the inner housing is a substantially enclosed structure; and
c) cells of interest, wherein the cells of interest are enclosed inside the inner housing.

35. The implantable therapeutic device according to claim 34, wherein the outer housing is made of a formable material comprising PDMS, carbon fiber, polyethylene oxide polymers, ceramics, acrylonitrile, butadiene, styrene, acetates, acrylics, delrin, epoxy fiberglass, glass, kymar, mica, nylon, polyetheretherketone (PEEK), polyethylene terephthalate (PET), polypropylene, polystyrene, polyurethane, polyvinyl chloride, or any combination thereof.

36. The implantable therapeutic device according to claim 34, wherein at least part of the outer housing is coated with PDMS or wherein the complete outer housing is coated with PDMS.

37. The implantable therapeutic device according to claim 34, wherein the outer housing is made of material consisting essentially of PDMS.

38. The implantable therapeutic device according to claim 34, wherein the shape of the outer housing is selected from the group consisting of a ring, a coil, a hollow tube, a hollow sphere, and a hollow inflated oval.

39. The implantable therapeutic device according to claim 38, wherein the outer housing is a ring having an inner diameter of about 0.1 mm to about 40.0 mm and an outer diameter of about 0.1 mm-about 50.0 mm.

40. The implantable therapeutic device according to claim 38, wherein the outer housing is a ring having a thickness of about 0.1 mm to about 5.0 mm.

41. The implantable therapeutic device according to claim 34, further comprising an imaging device.

42. The implantable therapeutic device according to claim 41, wherein the imaging device is one or more NMR coils.

43. The implantable therapeutic device according to claim 34, wherein the inner housing is made of material comprising alginate, agar, collagen, gelatin, or any combination thereof.

44. The implantable therapeutic device according to claim 34, wherein the inner housing is made of alginate having a guluronic acid content of about 10.0% to about 85.0%.

45. The implantable therapeutic device according to claim 34, wherein the shape of the inner housing is selected from the group consisting of a ring, a coil, a tube, a tube, a sphere, and an inflated oval.

46. The implantable therapeutic device according to claim 34, wherein cells of interest are encapsulated in alginate beads made of alginate having a guluronic acid content of about 10% to about 40% or about 10% to about 85% by weight or about 30% to about 85% by weight.

47. The implantable therapeutic device according to claim 46, the alginate bead has a diameter of about 10 microns to about 1000 microns.

48. The implantable therapeutic device according to claim 46, wherein about 1×10² cells to about 1×10⁹ cells are encapsulated per alginate bead.

49. The implantable therapeutic device according to claim 34, further comprising a therapeutic agent selected from the group consisting of an anti-diabetic agent, a biguanide, a sulfonylurea, insulin, an insulin mimetic, an alpha-glucosidase inhibitor, an anti-inflammatory agent, or any combination thereof.

50. The implantable therapeutic device according to claim 34, wherein cells of interest are selected from insulin-secreting cells, pancreatic beta cells, lung cells, ovary cells, colon cells, kidney cells, prostate cells, pancreas cells, testes cells, cardiomyocytes, endothelial cells, epithelial cells, lymphocytes, mast cells, eosinophils, vascular intimal cells, hepatocytes, leukocytes including mononuclear leukocytes, stem cells, osteoclasts, chondrocytes, connective tissue cells, keratinocytes, melanocytes, liver cells, adipocytes, or any combination thereof

51. The implantable therapeutic device according to claim 34, wherein said implantable therapeutic device comprises an outer housing that comprises one or more flanges capable of supporting an inner housing.

52. The implantable therapeutic device according to claim 51, wherein said implantable therapeutic device comprises a support member disposed upon a flange of said outer housing.

53. The implantable therapeutic device according to claim 52, wherein said support member is permeable.

54. A method for making an implantable therapeutic device, comprising:
a) providing liquid polydimethylsiloxane (PDMS);
b) forming the liquid polydimethylsiloxane (PDMS) into an outer housing wherein at least part of the outer housing is an open hollow cavity;
c) providing a liquid alginate having a guluronic acid content of about 10% to 85%;
d) dispersing cells of interest within the liquid alginate mixture to form a alginate-cell suspension;
e) forming alginate beads encapsulating cells of interest from the alginate-cell suspension by exposure to divalent cations;
f) mixing the alginate beads with a liquid alginate having a guluronic acid content of about 10% to 80%; and
g) filling the outer housing with the alginate beads/alginate mixture and spinning the outer housing until the alginate gels upon exposure to divalent cations to form an inner core;
wherein the inner housing is situated inside the open hollow cavity of the outer housing, and wherein the alginate beads encapsulating cells of interest are enclosed inside the inner housing.

55. A method of treating diabetes comprising implanting a device according to claim 34 into a subject having diabetes, wherein said cells of interest secrete insulin.

56. The method according to claim 55, wherein said cells have been transformed with a gene encoding insulin.

57. The method according to claim 55, wherein said cells are pancreatic cells.

58. A method of increasing levels of a protein of interest in a subject comprising implanting a device according claim 34 into a subject, wherein said cells of interest secrete a protein of interest from said device into said subject.

59. The method according to claim 58, wherein said protein of interest is insulin, a nerve growth factor, a growth factor, an interleukin, an interferon, a tumor necrosis factor, a parathyroid hormone, an erythropoietin, albumins, or transferrin.

60. The method according to claim 58, wherein said protein of interest is IL-1α, IL-β3, IL-2, IL-3, IL-4, IL-5, IL-6, IL-7, IL-8, IL-9, IL-10, IL-11, IL-12elasti, IL-13, IL-15, Il-16, Il-18, IL-18Bpa, IL-23, IL-24, VIP, erythropoietin, GM-CSF, G-CSF, M-CSF, platelet derived growth factor, TNF-α; TNF-β; MSF, FLT-3 ligand, EGF, fibroblast growth factor; aFGF; bFGF; FGF-3; FGF-4; FGF-5; FGF-6; FGF-7; insulin-like growth factor 1; IGF-2; vascular endothelial growth factor; IFN-γ; IFN-α; IFN-β; nerve growth factor; leukemia inhibitory factor; ciliary neurotrophic factor; oncostatin M; stem cell factor; TGF-α; TGF-β1; TGF-β2; LIGHT/TNFSF14 ; sTALL-1/TNFSF13B,TWEAK or a chemokine selected from the group consisting of BCA-1/BLC-1, BRAK/Kec, CXCL16, CXCR3, ENA-78/LIX, Eotaxin-1, Eotaxin-2/MPIF-2, Exodus-2/SLC, Fractalkine/Neurotactin, GROalpha/MGSA, HCC-1, I-TAC, Lymphotactin/ATAC/SCM, MCP-1/MCAF, MCP-3, MCP-4, MDC/STCP-1/ABCD-1, MIP-1α, MIP-1β, MIP-2α/GROβ, MIP-3α/Exodus/LARC, MIP-3β/Exodus-3/ELC, MIP-4/PARC/DC-CK1, PF-4, RANTES, SDF1α, TARC, and TECK.

61. A method of treating an endocrine disorder comprising the implantation of an implantable device according to claim 34 into an individual having an endocrine disorder, wherein said implantable device provides endocrine molecules selected from norepinephrine; epinephrine; dopamine; thyroid hormones, 3,5,3′-triiodothyronine (T3); 3,5,3′,5′-tetraiodothyronine (thyroxine, T4); follicle stimulating hormone (FSH), thyroid stimulating hormone (TSH), endorphins, luteinizing hormone (LH), prolactin, growth hormone, parathyroid hormone, leptin, ghrelin, cortisol, corticosterone, testosterone, melanocyte stimulating hormone, adrenocorticotropic hormone (ACTH), oxytocin, antidiuretic hormone, aldosterone, dehydroepiandrosterone (DHEA), DHEA sulfate (DHEA-S) glucocorticoids, mineralocorticoids, androgens, estrogens or progestagens.

62. The method according to claim 61, wherein said endocrine molecules are secreted from isolated tissue cells that secrete these molecules or by cells genetically engineered to secrete the endocrine molecules.

63. The method according to claim 61, wherein said implantable device is loaded with purified endocrine molecules.
